(19) United States
(12) Patent Application Publication
(10) Pub. No.: US 2025/0069364 A1
(43) Pub. Date: Feb. 27, 2025

(54) METHOD AND APPARATUS FOR MATCHING POINT CLOUD AND CT IMAGE

(71) Applicant: INJE UNIVERSITY INDUSTRY-ACADEMIC COOPERATION FOUNDATION, Gimhae-si, Gyeongsangnam-do (KR)

(72) Inventors: Moon Jun SOHN, Seoul (KR); Haenghwa LEE, Seoul (KR)

(73) Assignee: INJE UNIVERSITY INDUSTRY-ACADEMIC COOPERATION FOUNDATION, Gimhae-si, Gyeongsangnam-do (KR)

(21) Appl. No.: 18/684,069

(22) PCT Filed: Aug. 19, 2022
(86) PCT No.: PCT/KR2022/012457

§ 371 Date: Feb. 15, 2024

(30) Foreign Application Priority Data
Aug. 19, 2021 (KR) 10-2021-0109294

(51) Int. Cl. G06V 10/75; G06T 7/13; G06T 7/33; G06T 7/73; G06V 10/25

(52) U.S. Cl. CPC G06V 10/751; G06T 7/13; G06T 7/337; G06T 7/74; G06V 10/25; G06T 2200/04; G06T 2207/10028; G06T 2207/10081; G06V 2201/03

(57) ABSTRACT

An apparatus for matching a point cloud and a CT image according to one embodiment disclosed in the present document obtains target point cloud data from a surface image of an object photographed by a 3D camera; and matches the obtained target point cloud data with reference point cloud data by using normal distribution transform, wherein the reference point cloud data refers to data obtained from previous point cloud of the object or a CT image of the object, and on the basis of the matching, transformation information between the target point cloud data and the reference point cloud data may be provided.

### TECHNICAL FIELD

Embodiments disclosed in the present document relate to a technology of matching a point cloud of an object and a CT image, and more particularly, to a technology of matching a point cloud and a CT image using a normal distribution transform method.

### BACKGROUND ART

In the hypofractionated stereotactic radiosurgery (hypofractionated SRS or SBRT), the most important factor is to set a patient's stereotactic position on an operating table to obtain successful treatment results. It is important to accurately and reproducibly set a patient for success of hypofractionated stereotactic body radiotherapy. A radiation generator equipped with a medical diagnostic apparatus such as x-ray and CT is used to ensure accurate target localization of an accurate lesion. However, such lesion localizations have problems in that it is difficult to acquire and adjust images in real time and additional radiation exposure is required.

In order to solve this problem, a body localization system using a real-time body surface image is being developed. Specifically, a surface guided localization method is a noninvasive and real-time monitoring method which has recently received the most attention. However, the surface guided localization methods developed up to now are based on an iterative closest point (ICP) algorithm and it has problems in that it takes a long time due to iterative computation and a lot of data storage spaces are required. Further, the ICP algorithm based surface guided localization methods are greatly affected by surrounding noises and are sensitive to the error occurrence according to an abnormal value. Accordingly, development of surface guided localization methods to solve the above-mentioned problems is necessary.

### DISCLOSURE

### Technical Problem

The present disclosure is to provide a technique of matching a point cloud and a CT image based on a normal distribution transform algorithm.

### Technical Solution

A method for matching a point cloud and a CT image according to an embodiment includes obtaining target point cloud data from a surface image of an object photographed by a 3D camera: matching the obtained target point cloud data with reference point cloud data obtained from at least one CT image of the object by using normal distribution transform, in which the reference point cloud data refers to data obtained from previous point cloud of the object or a CT image of the object, and providing transformation information between the target point cloud data and the reference point cloud data, on the basis of matching.

In the method for matching a point cloud and a CT image according to an embodiment, the matching may include selecting point cloud data corresponding to a region of interest (RoI) from each of the reference point cloud data and the target point cloud data: obtaining a probability density function of the selected reference point cloud data; and calculating information about a matching degree of the selected target point cloud data and the obtained probability density function using a transformation estimate.

In the method for matching a point cloud and a CT image according to an embodiment, the selecting of point cloud data corresponding to the RoI may include: removing data having an outlier from each of the reference point cloud data and the target point cloud data; and selecting point cloud data corresponding to RoI from each of the reference point cloud data and the target point cloud data from which data having a outlier is removed.

In the method for matching a point cloud and a CT image according to an embodiment, in the providing of transformation information, 6 degree of freedom (6DoF) transformation information of the selected target point cloud data based on the selected reference point cloud data may be provided, on the basis of calculated information about the matching degree.

In the method for matching a point cloud and a CT image according to an embodiment, the providing of transformation information may include: providing point cloud data obtained by transforming the selected target point cloud data according to 6DoF transformation information.

The method for matching a point cloud and a CT image according to an embodiment may further include extracting a contour from a 3D image of the object by performing segmentation or edge extraction on the CT image of the object; and obtaining reference point cloud data of the object from the extracted contour.

An apparatus for matching a point cloud and a CT image according to an embodiment further includes an input unit, an output unit, a memory; and at least one processor. The at least one processor executes n instruction stored in the memory to obtain target point cloud data from a surface image of an object photographed by a 3D camera through the input unit: matches the obtained target point cloud data with reference point cloud data of the object by using normal distribution transform, the reference point cloud data refers to data obtained from previous point cloud of the object or a CT image of the object, and may provide transformation information between the target point cloud data and the reference point cloud data, on the basis of matching through the output unit.

A computer program product including a recording medium in which a program to perform a method for matching a point cloud and a CT image is stored includes a recording medium which stores a program to perform: an operation of obtaining target point cloud data from a surface image of an object photographed by a 3D camera: an operation of matching the obtained target point cloud data with reference point cloud data of the object by using normal distribution transform, in which the reference point cloud data refers to data obtained from previous point cloud of the object or a CT image of the object, and an operation of providing transformation information between the target point cloud data and the reference point cloud data, on the basis of matching.

### Advantageous Effects

According to the embodiments disclosed in the present document, the point cloud and the CT image are matched based on the normal distribution transform algorithm to lower the complexity of calculations and effectively reduce a memory amount used to store them.

### DESCRIPTION OF DRAWINGS

FIG. 1 is a flowchart for explaining a method for matching a point cloud and a CT image according to one embodiment of the present disclosure.

FIG. 2 is a view for explaining a method for obtaining reference point cloud data of an object according one embodiment.

FIG. 3 is a view for explaining a method for obtaining reference point cloud data of an object according to another embodiment.

FIG. 4 is a flowchart for more specifically explaining a method for matching a point cloud and a CT image according to one embodiment.

FIG. 5 is a view for explaining a method for matching a point cloud and a CT image using a normal distribution transform according to one embodiment.

FIG. 6 is a view for explaining a method for providing transformation information between a point cloud and a CT image by an electronic device according to one embodiment.

FIG. 7 is a block diagram of an electronic device according to one example embodiment.

### MODES OF THE INVENTION

The terms used in the present specification will be described briefly and the present invention will be described specifically.

Terminologies used in the specification are selected from general terminologies which are currently and widely used as much as possible while considering a function in the present invention, but the terminologies may vary in accordance with the intention of those skilled in the art, custom, or appearance of new technology. Further, in particular cases, the terms are arbitrarily selected by an applicant and in this case, the meaning thereof may be described in a corresponding section of the description of the disclosure. Therefore, the terminology used in the specification is analyzed based on a substantial meaning of the terminology and the specification rather than a simple title of the terminology.

In the specification, unless explicitly described to the contrary, the word “comprise” and variations such as “comprises” or “comprising,” will be understood to imply the inclusion of stated elements but not the exclusion of any other elements. In addition, the terms “-unit,” “-er,” “-or,” and “module” described in the specification mean units for processing at least one function and operation and may be implemented by hardware components or software components and combinations thereof.

A surface registration algorithm is a program which guides an object to receive the treatment in the same position at any time. In the present disclosure, target point cloud data representing a surface of a current object and point cloud data set from a previously obtained CT image are matched and transformation information according to the matching result is provided to guide a posture or a position of the object.

Hereinafter, the method for matching a point cloud and a CT image according to the present disclosure will be described in more detail with reference to Examples. However, the following Examples are set forth to illustrate the present disclosure, but the scope of the disclosure is not limited thereto.

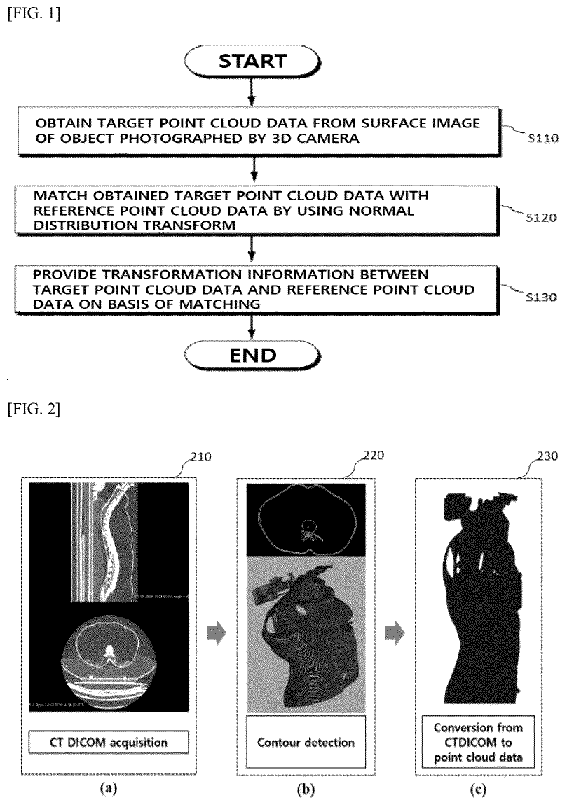

FIG. 1 is a flowchart for explaining a method for matching a point cloud and a CT image according to one embodiment of the present disclosure.

In step S110, an electronic device may obtain target point cloud data from a surface image of an object photographed by a 3D camera.

The electronic device may obtain a surface image of the object photographed by the 3D camera. For example, the electronic device may obtain a surface image of the object by photographing the object lying on an operating table using a 3D camera.

The electronic device may obtain point cloud data representing a surface of the object from the surface image. Hereinafter, point cloud data obtained based on the surface image of the object photographed by the 3D camera will be explained as target point cloud data for the sake of convenience. The target point cloud is a set cloud of plurality of points of a surface of the object spread out in a 3D space and position information of the points may be included in the target point cloud data.

The 3D camera may be included as a component of the electronic device or may be provided as a separate component at the outside of the electronic device and the 3D camera may include a Lidar sensor or an RGB-D sensor. However, these are merely illustrative, and a depth sensing sensor included in the 3D camera is not limited to the above-described type.

In step S120, the electronic device may match the obtained target point cloud data and reference point cloud data using normal distribution transform.

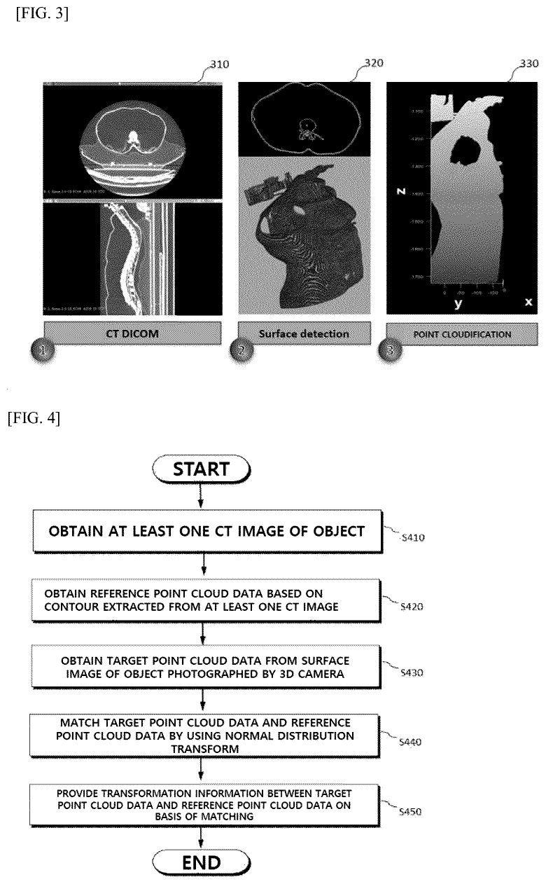

The reference point cloud data according to one embodiment may be obtained from a contour of the object recognized based on the predetermined algorithm from at least one CT image of the object. This will be described with reference to FIGS. 2 and 3.

Further, according to another embodiment, the reference point cloud data may refer to a previous point cloud of the object.

The electronic device according to the embodiment may select point cloud data corresponding to a region of interest (RoI) from each of the reference point cloud data and the target point cloud data. For example, the electronic device sets a rectangular surface area of approximately 600×380×160 mm³ of an object's abdomen as an RoI and may select reference point cloud data and target point cloud data included in the RoI from the entire reference point cloud data and target point cloud data.

However, this is merely illustrative, and the electronic device may select reference point cloud data and target point cloud data included in the RoI, after performing a preprocessing process of removing outliers on the reference point cloud data and the target point cloud data.

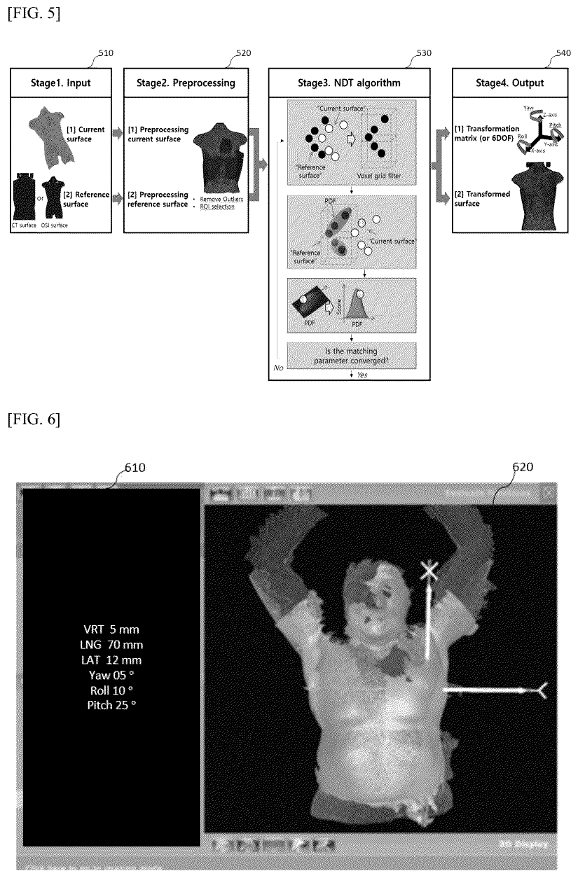

The electronic device may obtain a probability density function of the selected reference point cloud data. When the reference point cloud data is allocated to a 3D grid which is a normal distribution voxel (ND voxel), the probability density function may include a probability density function of each ND voxel. A method for calculating a probability density function of each ND voxel will be described in detail with reference to FIG. 5.

The electronic device may calculate information about a matching degree of the selected target point cloud data and the obtained probability density function, using a transformation estimate. The transformation estimate may be set to each of six degrees of freedom (DoF).

In step S130, the electronic device may provide transformation information between the target point cloud data and the reference point cloud data on the basis of the matching.

The electronic device may select a transformation estimate having a highest matching degree, based on information about the matching degree which is calculated for every transformation estimate. The electronic device according to one embodiment may provide a 6DoF value included in the selected transformation estimate as transformation information. Further, the electronic device according to another embodiment may provide point cloud data obtained by transforming the selected target point cloud data according to the transformation estimate.

In the meantime, the electronic device may perform the operations of the above-described steps S110 to S130 in real time. For example, the electronic device photographs the object in real time using a 3D camera to obtain the target point cloud data. The electronic device provides transformation information of the object on the basis of the obtained target point cloud data in real time to perform radiosurgery and treatment on the exact position of the object.

FIG. 2 is a view for explaining a method for obtaining reference point cloud data of an object according one embodiment.

The electronic device according to one embodiment may obtain at least one CT image of the object. The at least one obtained CT image may be stored as a digital imaging communications in medicine (DICOM).

The electronic device may obtain contour data 220 of the object by applying a segmentation method or an edge method to at least one CT DICOM image 210. The contour data 220 may be obtained by storing a voxel including a contour in a binary cube as a value which is not 0.

The electronic device may obtain information about a position of a voxel having a value which is not 0, among voxels which configure the contour data 220 as reference point cloud data 230.

FIG. 3 is a view for explaining a method for obtaining reference point cloud data of an object according to another embodiment.

As described above with reference to FIG. 2, the electronic device may obtain at least one CT image of the object. The at least one obtained CT image may be stored as a digital imaging communications in medicine (DICOM). The electronic device may obtain contour data 320 of the object by applying a segmentation method or an edge method to at least one CT DICOM image 310. The contour data 320 may be obtained by storing a voxel including a contour in a binary cube as a value which is not 0.

The electronic device may obtain surface temperature data of the object in addition to the contour data 320. The surface temperature data may be obtained as a result of photographing an object with a thermal imaging camera.

In the meantime, the electronic device may obtain the reference point cloud data 330 by combining information about a position of a voxel having a value which is not 0, among voxels which configure the contour data 320 and a temperature value corresponding to the voxel having a value which is not 0.

FIG. 4 is a flowchart for more specifically explaining a method for matching a point cloud and a CT image according to one embodiment.

In step S410, the electronic device may obtain at least one CT image of the object.

In step S420, the electronic device may obtain reference point cloud data on the basis of a contour extracted from at least one CT image. For example, the electronic device extracts a contour of the object by applying the segmentation method or the edge method and may obtain the reference point cloud data from the extracted contour.

In step S430, the electronic device may obtain target point cloud data from a surface image of an object photographed by a 3D camera.

In step S440, the electronic device may match the target point cloud data and reference point cloud data using normal distribution transform.

The electronic device may remove a value having an outlier from each of the target point cloud data and the reference point cloud data. The electronic device may specify an RoI in the target point cloud data and the reference point cloud data from which a value having an outlier is removed. The electronic device may select the target point cloud data and the reference point cloud data corresponding to the specified RoI. The electronic device may perform the matching of the selected target point cloud data and reference point cloud data using the normal distribution transform.

The normal distribution transform is a method for calculating a matching degree using distribution between points. According to the normal distribution transform, when the reference data is generated, raw data is not stored as a point cloud, but is calculated as an overall distribution value. That is, when the images are matched using the normal distribution transform, the correspondence relationship between points and distribution chart without repeated point-to-point calculations is calculated, so that the complexity of calculation formula is low and an amount of memories for storing it may be very efficiently reduced.

In step S450, the electronic device may provide transformation information between the target point cloud data and the reference point cloud data on the basis of the matching. For example, the electronic device may provide 6DoF which is a transformation difference between the translation and rotation matrix of target point cloud data with respect to the reference point cloud data as transformation information. Further, the electronic device may provide a result of transforming the target point cloud data according to the transformation information of 6DoF.

FIG. 5 is a view for explaining a method for matching a point cloud and a CT image using a normal distribution transform according to one embodiment.

In an input step 510, target point cloud data obtained as a result of photographing a current object and reference point cloud data obtained from a previously photographed CT image may be input to the electronic device.

In a preprocessing step 520, outliers may be removed from the target point cloud data and the reference point cloud data. Further, in the preprocessing step 520, after removing the outliers, target point cloud data and reference point cloud data belonging to the RoI may be selected.

In the normal distribution transforming step 530, a probability density function for the selected reference point cloud data may be obtained. For example, when the reference point cloud data is given as a 3D coordinate as represented in the following Equation 1 and a specific grid M has k voxels, it may be assumed that there are Mk 3D points.

$${x}_{i} = {( {x}_{i} , {y}_{i} , {z}_{i} )}^{t} ( i = 0 … M - 1 ) ( reference data ) \qquad [ Equation 1 ]$$

A mean and a variance of a normal distribution of the grid may be obtained on the basis of Equations 2 and 3 below.

$$Mean of grid {p}_{k} = \frac{1}{{M}_{k}} {\sum }_{i = 0}^{{M}_{k} - 1} {x}_{ki} \qquad [ Equation 2 ]$$

$$Variance of grid {\sum }_{k} = \frac{1}{{M}_{k}} {\sum }_{i = 0}^{{M}_{k} - 1} ( {x}_{ki} - {p}_{k} ) {( {x}_{ki} - {p}_{k} )}^{′} \qquad [ Equation 3 ]$$

A probability density function of the grid may be obtained on the basis of Equations 2 and 3 as represented in Equation 5 below.

$$e ( x ) ∼ exp ( - \frac{{( x - {p}_{k} )}^{′} {\sum }_{k}^{- 1} ( x - {p}_{k} )}{2} ) \qquad [ Equation 5 ]$$

The electronic device may search for appropriate coordinate transformation parameters and use the Newton optimization method to maximize the sum of probability densities corresponding to the target point cloud data as represented in Equation 6 below.

$$E ( X , t ) = {\sum }_{i}^{N - 1} exp \frac{- {( {x}_{i}^{′} - {p}_{i} )}^{t} {\sum }_{i}^{- 1} ( {x}_{i} - {p}_{i} )}{2} \qquad [ Equation 6 ]$$

In an output step 540, the electronic device outputs 6DoF which is a transformation difference between the translation and rotation matrix of target point cloud data with respect to the reference point cloud data, as transformation information. Further, the electronic device may output a result of transforming the target point cloud data according to the transformation information of 6DoF.

FIG. 6 is a view for explaining a method for providing transformation information between a point cloud and a CT image by an electronic device according to one embodiment.

Referring to FIG. 6, the electronic device may output 6DoF 610 which is a transformation difference between the translation and rotation matrix of target point cloud data with respect to the reference point cloud data, as transformation information. Further, the electronic device may output a result 620 of transforming the target point cloud data according to the transformation information of 6DoF.

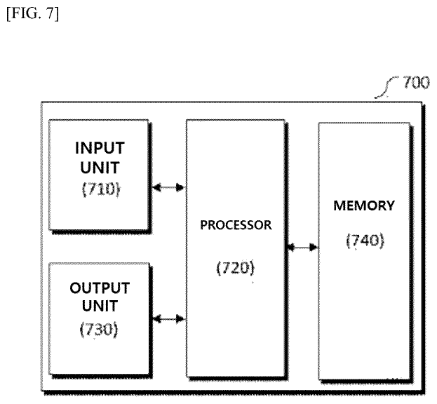

FIG. 7 is a block diagram of an electronic device 700 according to an example embodiment.

Referring to FIG. 7, the electronic device 700 may include an input unit 710, a processor 720, an output unit 730, and a memory 740. However, not all the illustrated components are essential components. The electronic device 700 may be implemented with components more than the illustrated components or may also be implemented with less components.

The input unit 710 may receive instructions or data to be used in a component of the electronic device 700 (for example, the processor 720) from the outside of the electronic device 700 (for example, a 3D camera).

The processor 720 may generally control the overall operation of the electronic device 700. For example, the processor 720 may obtain transformation information between the point cloud of the object and the CT image by executing the instructions stored in the memory 740.

The processor 720 may obtain the target point cloud data from a surface image of the object photographed by the 3D camera, through the input unit 710.

The processor 720 may match the obtained target point cloud data and the reference point cloud data using the normal distribution transform. The reference point cloud data refers to data obtained from a previous point cloud of the object or a CT image of the object.

The processor 720 may provide transformation information between target point cloud data and the reference point cloud data on the basis of the matching, through the output unit 730.

The processor 720 may select point cloud data corresponding to a region of interest (RoI) from each of the reference point cloud data and the target point cloud data. Further, the processor 720 may obtain a probability density function of the selected reference point cloud data. The processor 720 may calculate information about a matching degree of the selected target point cloud data and the obtained probability density function, using a transformation estimate. The processor 720 may provide 6DoF transformation information of the selected target point cloud data with respect to the selected reference point cloud data, on the basis of calculated information about the matching degree through the output unit 730.

The processor 720 may remove data having outliers from each of the reference point cloud data and the target point cloud data. The processor 720 may select point cloud data corresponding to RoI from each of the reference point cloud data and the target point cloud data from which data having a outlier is removed.

The processor 720 may provide point cloud data obtained by transforming the selected target point cloud data according to the 6DoF transformation information through the output unit 730.

The processor 720 performs segmentation or edge extraction on at least one CT image of the object to extract a contour from the 3D image of the object. The processor 720 may obtain reference point cloud data of the object from the extracted contour.

The output unit 730 may output 6DoF transformation information of the target point cloud data selected based on the reference point cloud data. Further, the output unit 730 may output point cloud data obtained by transforming the target point cloud data according to the 6DoF transformation information.

The memory 740 may store a program to allow the electronic device 700 to perform a method for matching a point cloud and a CT image according to the present disclosure. Further, the memory 740 may store reference point cloud data obtained from at least one CT image of the object.

The electronic device according to various embodiments disclosed in the present document may be various types of devices. For example, the electronic device may include medical equipment, etc. The electronic devices according to the embodiment of the present document are not limited to the above-described devices.

Examples of this document and terms used therein are not intended to limit the technical features described in the present disclosure to specific embodiments, and should be understood to include various modifications, equivalents, or substitutes of the embodiments. With regard to the description of drawings, like reference numerals denote like or related components. The singular form of a noun corresponding to an item may include one or more of the above items, unless the relevant context clearly indicates otherwise. As used herein, each of the phrases such as “A or B,” “at least one of A and B,” “at least one of A or B,” “A, B or C,” “at least one of A, B and C,” and “at least one of A, B, or C” may include any one of the items listed together with the corresponding phrase, or all possible combinations thereof. Terms such as “1st,” “2nd”, or “first,” or “second” may be used simply to distinguish one element from another, but do not limit the components in another aspect (for example, importance or order). When one (for example, first) component is referred to as “coupled” or “connected” to another (for example, second) component, with or without the terms “functionally” or “communicatively,” it means that one component may be connected to the other component directly (for example: wired), wirelessly, or by means of a third component.

The term used in various embodiments of the present document “module” includes a unit implemented by hardware, software, or firmware and for example, may be exchangeably used with a term such as a logic, a logic block, a part, or a circuit. The module may be an integrally configured component, or a minimum unit of the component which performs one or more functions, or a part thereof. For example, according to an exemplary embodiment, the module may be implemented by an application-specific integrated circuit (ASIC).

According to an exemplary embodiment, the method according to various exemplary embodiments disclosed in the present document may be provided to be included in a computer program product.

All documents, including published documents, patent applications, patents, etc., cited in the present invention may be incorporated into the present invention in the same manner as if each cited document was individually and specifically incorporated or as if it were incorporated as a whole in the present invention.

For understanding of the present invention, reference numerals are denoted in the exemplary embodiments illustrated in the drawings, and specific terms are used to describe the embodiments of the present invention. However, the present invention is not limited by the specific terms, and the present invention may include all components commonly conceived by those skilled in the art.

The present invention may be represented with functional block configurations and various processing steps. The functional blocks may be implemented by various numbers of hardware and/or software configurations which execute specific functions. For example, the present invention may employ direct circuit configurations such as a memory, a processing, a logic, or a look-up table in which various functions are executable by the control of one or more microprocessors or the other control devices.

Similar to execution of the components of the present disclosure with software programming or software elements, the present invention may be implemented by programming or scripting languages such as C, C++, Java, assembler including various algorithms implemented by a combination of data structures, processes, routines, or other program configurations. The functional aspects may be implemented by an algorithm executed in one or more processors. Further, the present invention may employ the related art for the electronic environment setting, signal processing and/or data processing. The terms such as “mechanism”, “element”, “unit”, and “configuration” are broadly used and are not limited to mechanical and physical configurations. The terms may include meaning of a series of routines of a software in association with the processor.

The specific implementations described in the present invention are examples and do not limit the scope of the present invention in any way. For simplicity of the specification, the description of another functional aspects of the electronic configurations, control systems, software, and the systems of the related art may be omitted. Further, connections of components illustrated in the drawing with lines or connection members illustrate functional connection and/or physical or circuit connections. Therefore, in the actual apparatus, it is replaceable or represented as additional various functional connections, physical connections, or circuit connections. Unless specifically stated as “essential,” “importantly,” it may not be an essential configuration to apply the present disclosure.

In the specification (specifically, claims) of the present disclosure, the terminology “said” or a similar terminology may correspond to both the singular form and the plural form. In addition, when a range is described in the present disclosure, individual values constituting the range are described in the detailed description of the present invention as including the invention to which the individual values within the range are applied (unless the context clearly indicates otherwise). Finally, the steps which configure the method according to the present invention may be performed in an appropriate order, unless specifically identified as an order of performance. The present invention is not necessarily limited by the described order of the steps. In the present disclosure, all examples, or exemplary terms (for example, and the like) are simply used to describe the present disclosure in detail so that if it is not limited by the claims, the scope of the present disclosure is not limited by the examples or the exemplary terms. Further, those skilled in the art may appreciate that various modifications, combinations, and changes may be made in accordance with the design conditions and factors within the scope of the appended claims or equivalents thereof.

1. A method for matching a point cloud and a CT image, comprising:
obtaining target point cloud data from a surface image of an object photographed by a 3D camera;
matching the obtained target point cloud data with reference point cloud data by using normal distribution transform, wherein the reference point cloud data refers to data obtained from a previous point cloud of the object or a CT image of the object; and
providing the transformation information between the target point cloud data and the reference point cloud data, on the basis of the matching.

2. The method of claim 1, wherein the matching includes:
selecting point cloud data corresponding to a region of interest (RoI) from each of the reference point cloud data and the target point cloud data;
obtaining a probability density function of the selected reference point cloud data; and
calculating information about a matching degree of the selected target point cloud data and the obtained probability density function using a transformation estimate.

3. The method of claim 2, wherein the selecting of point cloud data corresponding to the RoI includes:
removing data having an outlier from each of the reference point cloud data and the target point cloud data; and
selecting point cloud data corresponding to the RoI, from the reference point cloud data and the target point cloud data from which the data having an outlier is removed.

4. The method of claim 2, wherein in the providing of transformation information, 6 degree of freedom (6DoF) transformation information of the selected target point cloud data based on the selected reference point cloud data is provided, on the basis of calculated information about the matching degree.

5. The method of claim 4, wherein the providing of the transformation information includes:
providing point cloud data obtained by transforming the selected target point cloud data according to 6DoF transformation information.

6. The method of claim 1, further comprising:
extracting a contour from a 3D image of the object by performing segmentation or edge extraction on the CT image of the object; and
obtaining reference point cloud data of the object from the extracted contour.

7. An apparatus for matching a point cloud and a CT image, comprising:
an input unit;
an output unit;
a memory; and
at least one processor, wherein the at least one processor executes an instruction stored in the memory to obtain target point cloud data from a surface image of an object photographed by a 3D camera through the input unit, match the obtained target point cloud data with reference point cloud data by using normal distribution transform, in which the reference point cloud data refers to data obtained from a previous point cloud of the object or a CT image of the object, and provide the transformation information between the target point cloud data and the reference point cloud data, on the basis of the matching through the output unit.

8. The apparatus of claim 7, wherein the at least one processor executes an instruction stored in the memory to select point cloud data corresponding to a region of interest (RoI) from each of the reference point cloud data and the target point cloud data, obtain a probability density function of the selected reference point cloud data; and calculate information about a matching degree of the selected target point cloud data and the obtained probability density function using a transformation estimate.

9. The apparatus of claim 8, wherein the at least one processor executes an instruction stored in the memory to remove data having an outlier from each of the reference point cloud data and the target point cloud data; and select point cloud data corresponding to the RoI, from the reference point cloud data and the target point cloud data from which the data having an outlier is removed.

10. The apparatus of claim 8, wherein the at least one processor executes an instruction stored in the memory to provide 6DoF transformation information of the selected target point cloud data based on the selected reference point cloud data, on the basis of calculated information about the matching degree through the output unit.

11. The apparatus of claim 10, wherein the at least one processor executes an instruction stored in the memory to provide point cloud data obtained by transforming the selected target point cloud data according to 6DoF transformation information through the output unit.

12. The apparatus of claim 7, wherein the at least one processor executes an instruction stored in the memory to extract a contour from a 3D image of the object by performing segmentation or edge extraction on the CT image of the object and obtain reference point cloud data of the object from the extracted contour.

13. A computer program product including a recording medium in which a program to perform a method for matching a point cloud and a CT image is stored, comprising a recording medium which includes a program to perform:
an operation of obtaining target point cloud data from a surface image of an object photographed by a 3D camera;
an operation of matching the obtained target point cloud data with reference point cloud data by using normal distribution transform, wherein the reference point cloud data refers to data obtained from a previous point cloud of the object or a CT image of the object; and
an operation of providing the transformation information between the target point cloud data and the reference point cloud data, on the basis of the matching.
